(19) United States
(12) Patent Application Publication
(10) Pub. No.: US 2024/0363199 A1
(43) Pub. Date: Oct. 31, 2024

(54) METHOD USING MICROBIOME DATA FOR DIAGNOSING MEDICAL CONDITIONS

(71) Applicant: Cardiai Technologies, Calgary (CA)

(72) Inventors: MINAL BORKAR TRIPATHI, Calgary (CA); Sidharth Singh Bhinder, Calgary (CA); Anmol Singh Kapoor, Calgary (CA); Amrita Kaur, Calgary (CA)

(21) Appl. No.: 18/646,962

(22) Filed: Apr. 26, 2024

Related U.S. Application Data
(60) Provisional application No. 63/498,616, filed on Apr. 27, 2023.

(51) Int. Cl. G16B 30/00; G16B 40/00; G16H 50/20

(52) U.S. Cl. CPC G16B 30/00; G16B 40/00; G16H 50/20

(57) ABSTRACT

The present disclosure relates to a method (100) for diagnosing a medical condition using microbiome data. The method (100) includes collecting a sample from a subject and obtaining microbiome data from the collected sample. The method (100) also includes analyzing the microbiome data to identify patterns associated with the medical condition selected from the group consisting of infectious diseases, inflammatory disorders, metabolic disorders, autoimmune diseases, gastrointestinal disorders, and neurological disorders. The method (100) also includes identifying specific microbial taxa that are associated with the medical condition to determine patterns. The method (100) also includes comparing identified patterns with a reference set of patterns associated with the medical condition. The method (100) also includes using the comparison results indicating significant difference from the reference set of patterns to diagnose the medical condition.

### CROSS-REFERENCE TO RELATED APPLICATIONS

This application claims the benefit of U.S. Provisional Application 63/498,616 titled “A METHOD USING MICROBIOME DATA FOR DIAGNOSING MEDICAL CONDITIONS” filed by the applicant on Apr. 27, 2023, which is incorporated herein by reference in its entirety.

### FIELD OF THE INVENTION

Embodiments of the present invention relate to the field of diagnostic methods and more particularly, relate to a method for diagnosing medical condition using microbiome data.

### DESCRIPTION OF THE RELATED ART

Microbiomes are communities of microorganisms, including bacteria, viruses, fungi, and archaea, that inhabit various environments, such as the human gut, skin, mouth, and other body sites. Microbiome research is a rapidly evolving field, and there is still much to be discovered about the complex interactions between the microbiome and human health. However, recent advancements in technology have enabled researchers to collect and analyze large amounts of microbiome data from various parts of the body, such as the gut, skin, and oral cavity. This has led to the identification of specific bacterial strains and their functions, as well as the development of tools for analyzing the microbiome at a molecular level.

The microbiome has been linked to a wide range of conditions, including chronic diseases such as inflammatory bowel disease, diabetes, and cancer, as well as neurological disorders such as depression and autism. Researchers are also exploring the role of the microbiome in maintaining overall health and preventing disease, such as its impact on immune system function and the development of allergies.

In addition to its potential as a diagnostic tool and therapeutic target, the microbiome is also being investigated for its use in drug discovery. Microbes produce a vast array of bioactive molecules, many of which have the potential to be developed into drugs. Furthermore, the microbiome can influence the efficacy and toxicity of existing drugs, providing opportunities for personalized medicine and drug optimization.

Though, using microbiome data to diagnose medical conditions has numerous advantages but it is also riddled several challenges. As microbiomes are incredibly diverse, with thousands of species present in various proportions deciphering meaningful patterns from this complexity is the most significant challenge, which makes it exceptionally complex to correlate specific microbiome compositions with human health or disease.

Further adding to incredible diversity, microbiomes can vary significantly between individuals due to numerous reasons like genetics, diet, lifestyle, and environmental factors. Such variability makes it difficult to establish universal standard for specific medical conditions. Additionally, collecting and processing microbiome sample requires specialized equipment, techniques and training.

In addition to this, the microbiomes can change over time due to various factors, such as diet, medications, infections, and age. Understanding the temporal variability of microbiomes and their relevance to human health and disease adds another layer of complexity to data analysis and interpretation of microbiomes data.

Overall, the microbiome represents a promising avenue for improving human health and developing novel therapies. However, there is still much to be learned about the intricacies of the microbiome and its role in disease. As such, continued research and development in this field are essential for unlocking the full potential of the microbiome for human health.

Accordingly, to overcome the disadvantages of the prior art, there is an urgent need for a technical solution that overcomes the above-stated limitations in the prior arts. The present invention provides a method using microbiome data for diagnosing medical conditions.

### SUMMARY OF THE INVENTION

The present disclosure solves all the above major limitations of a method using microbiome data for diagnosing medical conditions. Further, the present disclosure ensures that the disclosed invention may fulfil following objectives.

An objective of the present disclosure is to develop a method using microbiome data for diagnosing medical conditions in reliable and efficient manner.

Another objective of the present disclosure is to develop a method for diagnosing medical conditions using microbiome data that can accurately identify microbial signatures or biomarkers associated with specific medical conditions.

Another objective of the present disclosure is to develop a method for diagnosing medical conditions using microbiome data that has high discriminatory power to minimize false positives and negatives.

Another objective of the present disclosure is to develop a method for diagnosing medical conditions using microbiome data that can be used across diverse populations, sample types, and experimental conditions.

Yet another objective of the present disclosure is to develop a method for diagnosing medical conditions using microbiome data that can analyze large volumes of microbiome data efficiently and be scalable.

Yet another objective of the present disclosure is to develop a method for diagnosing medical conditions using microbiome data that can seamlessly integrate with existing clinical workflows to facilitates widespread.

Embodiments of the present invention relate to a method for diagnosing a medical condition using microbiome data. The method includes collecting a sample from a subject and obtaining microbiome data from the collected sample. The method also includes analyzing the microbiome data to identify patterns associated with the medical condition selected from the group consisting of infectious diseases, inflammatory disorders, metabolic disorders, autoimmune diseases, gastrointestinal disorders, and neurological disorders. The method also includes identifying specific microbial taxa that are associated with the medical condition to determine patterns. The method also includes comparing identified patterns with a reference set of patterns associated with the medical condition. The method also includes using the comparison results indicating significant difference from the reference set of patterns to diagnose the medical condition.

In accordance with an embodiment of the present invention, the microbiome data analysis generates taxonomic and/or functional profiles of microbial communities present in the microbiome data obtained from the sample collected.

In accordance with an embodiment of the present invention, the analysis is performed using the 16S rRNA sequencing or metagenomic sequencing.

In accordance with an embodiment of the present invention, the sample comprises a stool sample, a blood sample, a tissue sample or a sample of other bodily fluids.

In accordance with an embodiment of the present invention, the reference set of patterns includes microbiome data, reference profiles, and diagnostic information associated with the subject.

In accordance with an embodiment of the present invention, the method also includes monitoring the progression of the condition by using and analyzing changes in the microbiome data.

Another embodiment of the present invention, a computer implemented system of diagnosing a medical condition using microbiome data. The system comprising a sample collection and microbiome extraction module. The system also comprising a microbiome data pre-processing and storage module linked to the sample collection and microbiome extraction module and the microbiome data storage module configured to digitize, standardize and securely store microbial deoxyribonucleic acid (DNA), ribonucleic acid (RNA), or proteins present in the sample. The system also comprising a communication network linked to the microbiome data pre-processing and storage module and the communication network configured to enable interconnectivity between a plurality of components. The system also comprising an analytics and processing module linked to the microbiome data pre-processing module via the communication network and the microbiome data storage module further comprising a microbiome analysis unit. The microbiome data storage module also comprising a comparison unit. The microbiome data storage module also comprising an intelligent diagnosis unit. The system also comprising a database linked to the comparison unit via the communication network and the database configured to store a reference set of patterns associated with the medical condition. The system also comprising a user device linked to the intelligent diagnosis unit via the communication network and the user device configured for visualization of the results obtained from the intelligent diagnosis unit.

In accordance with an embodiment of the present invention, the microbiome analysis unit uses bioinformatics software for analyzing microbial deoxyribonucleic acid (DNA), ribonucleic acid (RNA), or proteins present in the sample.

In accordance with an embodiment of the present invention, the comparison unit is characterized by computational algorithms and data processing techniques for identifying correlations between microbial features in the microbiome data and known conditions.

In accordance with an embodiment of the present invention, the intelligent diagnosis unit is characterized as decision making unit employing machine learning models and/or diagnostic algorithms for determining the likelihood or probability of the subject having the condition based on the comparison results obtained from the comparison unit.

In accordance with an embodiment of the present invention, the intelligent diagnosis unit generates diagnostic report indicating the presence or absence of the condition in the subject based on the comparison.

In accordance with an embodiment of the present invention, the intelligent diagnosis unit may also comprise a feedback mechanism linked to the user device and the feedback mechanism configured for refining diagnostic algorithms.

In accordance with an embodiment of the present invention, the feedback mechanism configured for improving the accuracy of condition diagnosis based on feedback from clinical outcomes and healthcare professionals.

### BRIEF DESCRIPTION OF THE DRAWINGS

So that the manner in which the above-recited features of the present invention is understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

The invention herein will be better understood from the following description with reference to the drawings, in which:

FIG. 1 is a flowchart illustrating a method for diagnosing a medical condition using microbiome data, in accordance with one embodiment of the present invention; and

FIG. 2 is a computer-implemented system of diagnosing a medical condition using microbiome data, in accordance with an embodiment of the present invention.

The method and system of diagnosing a medical condition using microbiome data is illustrated in the accompanying drawings, which like reference letters indicate corresponding parts in the various figures. It should be noted that the accompanying figure is intended to present illustrations of exemplary embodiments of the present disclosure. This figure is not intended to limit the scope of the present disclosure. It should also be noted that the accompanying figure is not necessarily drawn to scale.

### DETAILED DESCRIPTION OF THE INVENTION

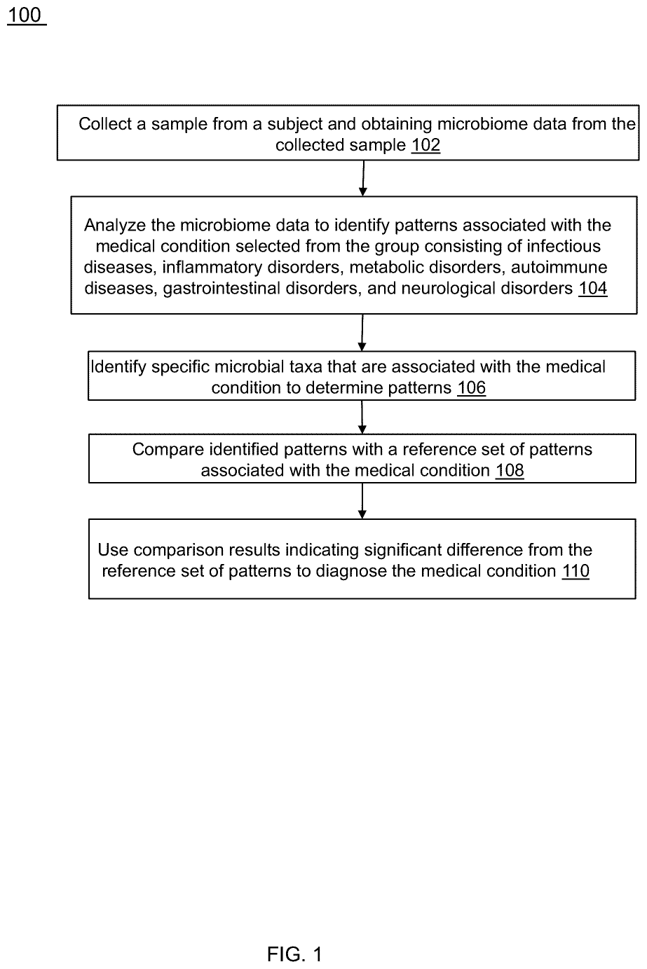

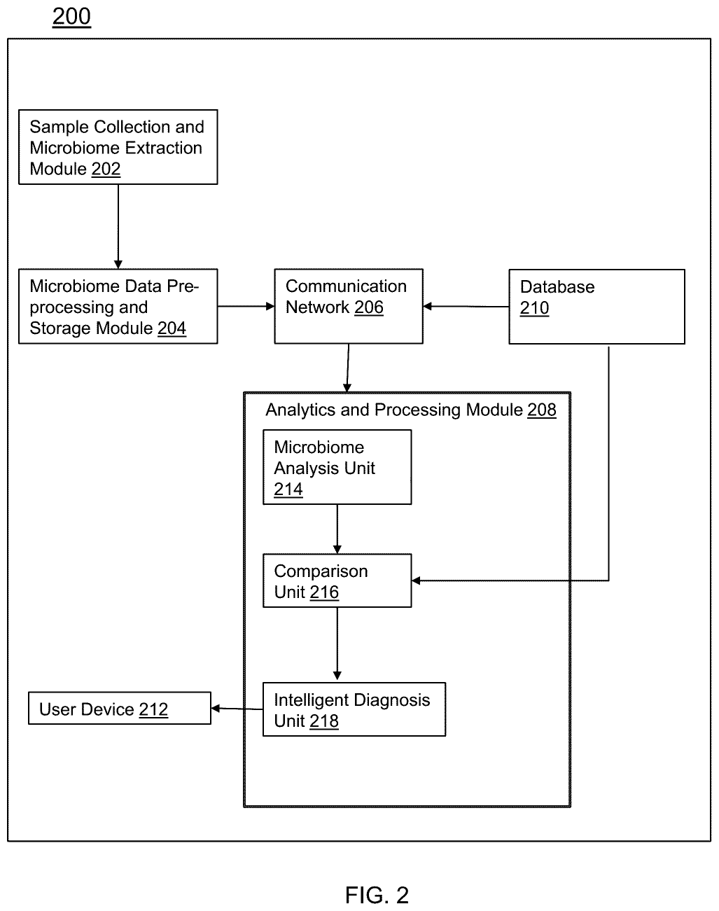

The principles of the present invention and their advantages are best understood by referring to FIGS. 1 and 2. In the following detailed description, numerous specific details are set forth in order to provide a thorough understanding of the embodiment of the invention as illustrative or exemplary embodiments of the invention, specific embodiments in which the invention may be practiced are described in sufficient detail to enable those skilled in the art to practice the disclosed embodiments. However, it will be obvious to a person skilled in the art that the embodiments of the invention may be practiced with or without these specific details. In other instances, well-known methods, procedures and components have not been described in detail so as not to unnecessarily obscure aspects of the embodiments of the invention.

The following detailed description is, therefore, not to be taken in a limiting sense, and the scope of the present invention is defined by the appended claims and equivalents thereof. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. References within the specification to “one embodiment,” “an embodiment,” “embodiments,” or “one or more embodiments” are intended to indicate that a particular feature, structure, or characteristic described in connection with the embodiment is included in at least one embodiment of the present invention.

Although the terms first, second, etc. may be used herein to describe various elements, these elements should not be limited by these terms. These terms are generally only used to distinguish one element from another and do not denote any order, ranking, quantity, or importance, but rather are used to distinguish one element from another. Further, the terms “a” and “an” herein do not denote a limitation of quantity, but rather denote the presence of at least one of the referenced items.

The conditional language used herein, such as, among others, “can,” “may,” “might,” “may,” “e.g.,” and the like, unless specifically stated otherwise, or otherwise understood within the context as used, is generally intended to convey that certain embodiments include, while other embodiments do not include, certain features, elements and/or steps.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

The following brief definition of terms shall apply throughout the present invention:

FIG. 1 is a flowchart illustrating a method 100 for diagnosing a medical condition using microbiome data, in accordance with one embodiment of the present invention. The method 100 may comprise following steps.

At 102, collecting a sample from a subject and obtaining microbiome data from the collected sample.

At 104, analyzing the microbiome data to identify patterns associated with the medical condition selected from the group consisting of infectious diseases, inflammatory disorders, metabolic disorders, autoimmune diseases, gastrointestinal disorders, and neurological disorders.

At 106, identifying specific microbial taxa that are associated with the medical condition to determine patterns.

At 108, comparing identified patterns with a reference set of patterns associated with the medical condition.

At 110, using the comparison results indicating significant difference from the reference set of patterns to diagnose the medical condition.

The microbiome data analysis may generate taxonomic and/or functional profiles of microbial communities present in the microbiome data obtained from the sample collected.

The analysis may be performed using the 16S rRNA sequencing or metagenomic sequencing.

The sample may comprise a stool sample, a blood sample, a tissue sample or a sample of other bodily fluids.

The reference set of patterns may include microbiome data, reference profiles, and diagnostic information associated with the subject.

The method may also include monitoring the progression of the condition by using and analyzing changes in the microbiome data.

In a preferred embodiment, the reference set may be generated from a control population, such as a healthy population. In a preferred embodiment, the method 100 may also be used to monitor the progression of the condition, as changes in the microbiome data can be used to assess the severity of the condition. In a preferred embodiment, the subject is a living being, such as, human, and non-human animal.

In some embodiments, the comparison of the microbiome data with the reference set may be performed using various bioinformatics tools and databases to identify correlations between microbial features and known conditions. In an embodiment of the present disclosure, the medical condition diagnosis may be determining the likelihood or probability of the subject having the condition based on the comparison results obtained.

In some embodiments, the method 100 may employ various tools available for the analysis of microbiomes including, but not limited to, QUIME (Quantitative Insights Into Microbial Ecology, mothur, MetaPhlAn (Metagenomic Phylogenetic Analysis), PICRUSt (Phylogenetic Investigation of Communities by Reconstruction of Unobserved States), LEfSe (Linear discriminant analysis Effect Size), DESeq2, STAMP (Statistical Analysis of Metagenomic Profiles0, Phyloseq, DADA2 and Kraken.

In some embodiments, for microbiome data analysis, the method 100 may employ various machine learning algorithms including, but not limited to, Random Forest, Support Vector Machines (SVM), Logistic Regression, Gradient Boosting Machines (GBM), Neural Networks such as, feedforward neural networks, convolutional neural networks (CNNs), and recurrent neural networks (RNNs), K-means Clustering, Principal Component Analysis (PCA) and Canonical Correlation Analysis (CCA).

FIG. 2 is a computer-implemented system 200 of diagnosing a medical condition using microbiome data, in accordance with an embodiment of the present invention.

The system 200 may comprise a sample collection and microbiome extraction module 202, a microbiome data pre-processing and storage module 204, a communication network 206, an analytics and processing module 208, a database 210, and a user device 212.

The sample collection and microbiome extraction module 202 may use standard protocols of collecting biological samples and preserving them to avoid contamination and preserve integrity of the collected sample. In an embodiment of the present disclosure, extraction of microbiome is performed by breaking down the sample matrix, separating microbial cells from other debris, and lysing the cells to release their genetic material. In some embodiments, for the extraction of microbiome data, techniques such as, but not limited to, bead beating, chemical lysis, or enzymatic digestion are used to break open cells and release nucleic acids.

The microbiome data pre-processing and storage module 204 may be linked to the sample collection and microbiome extraction module 202 and the microbiome data storage module 204 configured to digitize, standardize and securely store microbial deoxyribonucleic acid (DNA), ribonucleic acid (RNA), or proteins present in the sample.

In some embodiments, the data pre-processing and storage module 204 may remove contaminations from the microbiome data that could interfere with downstream analysis. In some embodiments, the data pre-processing and storage module 204 may ensure that the extracted microbiome data is of sufficient quantity and quality for downstream processing, as per the processing requirements. In some embodiments, the data pre-processing and storage module 204 may ensure accurate data interpretation of the physical microbiome and perform comprehensive categorization and linking to ensure reproducibility.

In an embodiment of the present disclosure, the data pre-processing and storage module 204 may use various sequencing platforms to generate waw sequence data from the extracted deoxyribonucleic acid (DNA) and ribonucleic acid (RNA). In some embodiments, the data pre-processing and storage module 204 may integrate the stored data with metadata, including sample characteristics, environmental variables, and clinical information.

The communication network 206 may be linked to the microbiome data pre-processing and storage module 204 and the communication network 206 configured to enable interconnectivity between a plurality of components.

In an embodiment of the present disclosure, the communication network 206 may include any networking technology such as, but not limited to, an Internet, a Local Area Network (LAN), a Wide Area Network (WAN), a Metropolitan Area Network (MAN), and so forth. In some embodiments of the present invention, the communication network 206 may include a wireless network, such as, but not limited to, a cellular network and may employ various technologies to provide seamless internet connectivity and enable high-speed data transfer.

In some embodiments of the present invention, the communication network 206 may employ various wireless communication technologies including, but not limited to, Code Division Multiple Access (CDMA), 2G communication technology, 3G communication technology, 4G communication technology, 5G communication technology, and long-term evolution (LTE). Embodiments of the present disclosure are intended to include or cover any type of the wireless communication technologies or networks, including known, related art, and/or later developed technologies.

The analytics and processing module 208 may be linked to the microbiome data pre-processing module 204 via the communication network 206 and the microbiome data storage module 208 further comprising a microbiome analysis unit 214, a comparison unit 216, and an intelligent diagnosis unit 218.

The microbiome analysis unit 214 may use bioinformatics software for analyzing microbial deoxyribonucleic acid (DNA), ribonucleic acid (RNA), or proteins present in the sample.

In a preferred embodiment, the analytics and processing module 208 may use 16S rRNA for bacteria and ITS for fungi as references for determining the taxonomic composition of the microbiome. In some embodiments, the microbiome analysis unit 214 may correlate the microbiome data with clinical data, biomarkers, or other diagnostic information to improve the diagnostic accuracy. In some embodiments, the microbiome analysis unit 214 may process the pre-processed sequence and use various bioinformatics tools to identify and classify microbial taxa present in the sample based on the DNA sequences.

In some embodiments, after taxonomic profiling the microbiome analysis unit 214 may use various bioinformatics software to predict the functional potential of microbial communities by annotating genes and pathways present in the sequencing data to gain understand on the metabolic capabilities and ecological roles of different microbial taxa within the community. In some embodiments, the microbiome analysis unit 214 may employ statistical methods to compare the abundance of microbial taxa or functional categories between different samples or experimental conditions to identify microbial features that are significantly associated with specific medical condition.

In some embodiments, the microbiome analysis unit 214 may use advanced bioinformatics approaches including machine learning algorithms to microbiome data for predictive modeling or classification tasks. For an instance, predicting disease outcomes based on microbial signatures or classifying samples into different environmental categories.

The comparison unit 216 may be characterized by computational algorithms and data processing techniques for identifying correlations between microbial features in the microbiome data and known conditions.

In some embodiments, the comparison unit 216 may identify statistically significant associations or correlations between microbial features such as, taxa or functional profiles and other variables. The comparison unit 216 may perform functions, such as linear regression, logistic regression, or such. In some embodiments, the comparison unit 216 may use advanced machine learning algorithms to compare complex patterns and relationships identified in the microbiome data with the reference sets of patterns stored in the database 210. In some embodiments, the comparison unit 216 may use machine learning models and diagnostic algorithms to perform feature selection to identify the relevant microbial features before comparing with the reference sets.

In some embodiments, the comparison unit 216 may use unsupervised learning techniques like clustering or dimensionality reduction to reveal inherent structures within the microbiome data before comparing with the reference sets. In some embodiments, the comparison unit 216 may identify microbial interactions and co-occurring taxa that are associated with specific medical conditions.

The intelligent diagnosis unit 218 may be characterized as decision making unit employing machine learning models and/or diagnostic algorithms for determining the likelihood or probability of the subject having the condition based on the comparison results obtained from the comparison unit 216.

The intelligent diagnosis unit 218 may generate diagnostic report indicating the presence or absence of the condition in the subject based on the comparison.

In some embodiments, the intelligent diagnosis unit 218 may use metadata integrated with the microbiome data to contextualize microbiome findings and identify correlations between microbial features and external factors and predict associated medical condition.

In some embodiments, the intelligent diagnosis unit 218 may use machine learning models such as, but not limited to, logistic regression, random forests, support vector machines, or deep learning architectures. In some embodiments, the intelligent diagnosis unit 218 may perform probability estimation to provide the probability or likelihood of an association with certain medical conditions. In some embodiments, the intelligent diagnosis unit 218 may integrate analyzed microbiome data with clinical information such as, symptoms, medical history, and laboratory results to improve diagnostic accuracy. In some embodiments, the intelligent diagnosis unit 218 may be capable of diagnostic interpretability using various techniques such as, but not limited to, SHAP (Shapley Additive explanations), and LIME (Local Interpretable Model-agnostic Explanations).

The database 210 may be linked to the comparison unit 216 via the communication network 206 and the database 210 configured to store a reference set of patterns associated with the medical condition.

In an embodiment of the present disclosure, the database 210 may have capabilities for managing large datasets and databases of microbial sequences including, but not limited to, storage, retrieval, and updating of reference databases used for taxonomic classification and functional annotation. In some embodiments, the database 210 may be, but not limited to, a centralized database, a distributed database, a personal database, an operational database, a relational database, a cloud database, an object-oriented database, and so on.

In some embodiments, the database 210 may store structured data representing microbial patterns associated with different medical conditions and/or healthy condition. In some embodiment, each record in the database 210 corresponds to a specific pattern, which may include microbial taxa abundances, functional profiles, or other relevant features extracted from microbiome data. In some embodiment, each pattern in the database 210 may be annotated with metadata describing the associated medical condition and relevant clinical information.

In an alternative embodiment, the database 210 may integrate microbiome data from diverse sources, including research studies, clinical trials, public repositories, and proprietary datasets. In some embodiments, the data from multiple studies provide a comprehensive reference set of patterns associated with the various medical condition of interest. In an alternative embodiment, the database 210 may support various query functionalities to retrieve reference patterns associated with various medical conditions or criteria.

The user device 212 may be linked to the intelligent diagnosis unit 218 via the communication network 206 and the user device 212 configured for visualization of the results obtained from the intelligent diagnosis unit 218.

In an embodiment of the present disclosure, the user device 212 may be capable of displaying the diagnostic results, recommendations, and visualizations based on the results obtained from the intelligent diagnosis unit 218. In some embodiments, the user device 212 may incorporate interactive visualization tools for displaying diagnostic results obtained. In some embodiments, user device 212 may be capable of integrating software tools used to visualize microbiome data in various formats such as, but not limited to, bar charts, heatmaps, or PCoA plots, for exploring patterns and relationships.

In some embodiments, the user device 212 may be any a user device may be any desktop computer, laptop computer, a user computer, tablet computer, any personal computer, any smart digital interactive screen cellular telephone, or a combination of any these data processing devices or any other data processing devices. The user device 212 may comprise, a graphical user interface, a memory, an input unit, a microprocessor/microcontroller, and capability to connect with internet via the communication network 206. Embodiments of the present disclosure are intended to include and/or otherwise cover any kind of user device including known, related/prior art, and/or later developed technologies.

The system 200 may also comprise a feedback mechanism linked to the user device

212 and the feedback mechanism configured for refining diagnostic algorithms and improving the accuracy of condition diagnosis based on feedback from clinical outcomes and healthcare professionals.

In an embodiment of the present disclosure, the user device 212 may be used by the healthcare professionals to input additional clinical information, review diagnostic results obtained, review and assess various components of the system 100 and provide feedback through the feedback mechanism. In an embodiment of the present disclosure, the user device 212 may be operationally coupled to the feedback mechanism that allow the healthcare professionals to provide feedback on the accuracy of the diagnostic results provided by the intelligent diagnosis unit 218.

In a best mode of operation, the method 100 may include obtaining microbiome data from a sample, and analyzing the microbiome data to identify patterns associated with the condition. The method 100 further comprises comparing the identified patterns with a reference set of patterns associated with the condition, and using the comparison to diagnose the condition.

The key advantage of the disclosed method 100 and system 200 is robustness in leveraging microbiome data for diagnosing medical conditions that can ultimately improve patient outcomes and healthcare delivery. The proposed method 100 is effective, reliable, and clinically relevant. Yet another advantage is capability of the method 100 to target specific medical conditions, distinguishing it from other similar conditions or healthy states. The method is sensitive enough to detect subtle variations in microbiome composition that may indicate disease presence or progression.

Another advantage is that the method ensures consistent results and that allows for easy comparisons with reference sets. Further, monitoring of microbiome data changes can track disease progression, treatment response, and recurrence risk. Use of predictive modeling based on microbiome data can help identify at-risk individuals and implement preventive interventions.

In conclusion, the method 100 may comprises obtaining microbiome data from a sample, and analyzing the microbiome data to identify patterns associated with the condition. The microbiome data may be obtained from a variety of sources, such as a stool sample, a blood sample, or a tissue sample. The microbiome data is then analyzed to identify patterns associated with the condition.

The analysis may be accomplished by utilizing a variety of techniques, such as 16S rRNA sequencing or metagenomic sequencing. Such techniques may be used to identify specific microbial taxa that are associated with the condition. The identified patterns may be compared with a reference set of patterns associated with the condition. The reference set may typically be generated from a control population, such as a healthy population. The comparison of the identified patterns with the reference set may be used to diagnose the condition. If the identified patterns may be significantly different from the reference set, then this may be used to diagnose the condition.

In a case that no conflict occurs, the embodiments in the present disclosure and the features in the embodiments may be mutually combined. The foregoing descriptions are merely specific implementations of the present disclosure, but are not intended to limit the protection scope of the present disclosure. Any variation or replacement readily figured out by a person skilled in the art within the technical scope disclosed in the present disclosure shall fall within the protection scope of the present disclosure. Therefore, the protection scope of the present disclosure shall be subject to the protection scope of the claims.

The foregoing descriptions of specific embodiments of the present technology have been presented for purposes of illustration and description. They are not intended to be exhaustive or to limit the present technology to the precise forms disclosed, and obviously many modifications and variations are possible in light of the above teaching. The embodiments were chosen and described in order to best explain the principles of the present technology and its practical application, to thereby enable others skilled in the art to best utilize the present technology and various embodiments with various modifications as are suited to the particular use contemplated. It is understood that various omissions and substitutions of equivalents are contemplated as circumstance may suggest or render expedient, but such are intended to cover the application or implementation without departing from the spirit or scope of the claims of the present technology.

What is claimed for:

1. A method for diagnosing a medical condition using microbiome data, the method comprising:
collecting a sample from a subject and obtaining microbiome data from the collected sample;
analyzing the microbiome data to identify patterns associated with the medical condition selected from the group consisting of infectious diseases, inflammatory disorders, metabolic disorders, autoimmune diseases, gastrointestinal disorders, and neurological disorders;
identifying specific microbial taxa that are associated with the medical condition to determine patterns;
comparing identified patterns with a reference set of patterns associated with the medical condition; and
using the comparison results indicating significant difference from the reference set of patterns to diagnose the medical condition.

2. The method of claim 1, wherein the microbiome data analysis generates taxonomic and/or functional profiles of microbial communities present in the microbiome data obtained from the sample collected.

3. The method of claim 2, wherein the analysis is performed using the 16S rRNA sequencing or metagenomic sequencing.

4. The method of claim 1, wherein the sample comprises a stool sample, a blood sample, a tissue sample or a sample of other bodily fluids.

5. The method of claim 1, wherein the reference set of patterns includes microbiome data, reference profiles, and diagnostic information associated with the subject.

6. The method of claim 1, wherein the method also includes monitoring the progression of the condition by using and analyzing changes in the microbiome data.

7. A computer implemented system of diagnosing a medical condition using microbiome data, the system comprising:
a sample collection and microbiome extraction module;
a microbiome data pre-processing and storage module linked to the sample collection and microbiome extraction module, the microbiome data storage module configured to digitize, standardize and securely store microbial deoxyribonucleic acid (DNA), ribonucleic acid (RNA), or proteins present in the sample;
a communication network linked to the microbiome data pre-processing and storage module, the communication network configured to enable interconnectivity between a plurality of components;
an analytics and processing module linked to the microbiome data pre-processing module via the communication network, the microbiome data storage module further comprising:
a microbiome analysis unit;
a comparison unit; and
an intelligent diagnosis unit;
a database linked to the comparison unit via the communication network, the database configured to store a reference set of patterns associated with the medical condition; and
a user device linked to the intelligent diagnosis unit via the communication network, the user device configured for visualization of the results obtained from the intelligent diagnosis unit.

8. The system of claim 7, wherein the microbiome analysis unit uses bioinformatics software for analyzing microbial deoxyribonucleic acid (DNA), ribonucleic acid (RNA), or proteins present in the sample.

9. The system of claim 7, wherein the comparison unit is characterized by computational algorithms and data processing techniques for identifying correlations between microbial features in the microbiome data and known conditions.

10. The system of claim 7, wherein the intelligent diagnosis unit is characterized as decision making unit employing machine learning models and/or diagnostic algorithms for determining the likelihood or probability of the subject having the condition based on the comparison results obtained from the comparison unit.

11. The system of claim 11, wherein the intelligent diagnosis unit generates diagnostic report indicating the presence or absence of the condition in the subject based on the comparison.

12. The system of claim 7, wherein the intelligent diagnosis unit may also comprise a feedback mechanism linked to the user device and the feedback mechanism configured for:
refining diagnostic algorithms; and
improving the accuracy of condition diagnosis based on feedback from clinical outcomes and healthcare professionals.
